SEQUENCE LISTING (16 sequences)

SEQ ID NO: 1 - protein, 108 aa, Artificial Sequence
  Description of Artificial Sequence Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 121 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 3 - protein, 5 aa, Artificial Sequence
  Description of Artificial Sequence Synthetic peptide
RYWMS

SEQ ID NO: 4 - protein, 17 aa, Artificial Sequence
  Description of Artificial Sequence Synthetic peptide
NIKQDGSEKYYVDSVKG

SEQ ID NO: 5 - protein, 12 aa, Artificial Sequence
  Description of Artificial Sequence Synthetic peptide
EGGWFGELAFDY

SEQ ID NO: 6 - protein, 12 aa, Artificial Sequence
  Description of Artificial Sequence Synthetic peptide
RASQRVSSSYLA

SEQ ID NO: 7 - protein, 7 aa, Artificial Sequence
  Description of Artificial Sequence Synthetic peptide
DASSRAT

SEQ ID NO: 8 - protein, 9 aa, Artificial Sequence
  Description of Artificial Sequence Synthetic peptide
QQYGSLPWT

SEQ ID NO: 9 - protein, 139 aa, Artificial Sequence
  Description of Artificial Sequence Synthetic polypeptide
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 10 - protein, 167 aa, Artificial Sequence
  Description of Artificial Sequence Synthetic polypeptide
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 11 - protein, 10 aa, Artificial Sequence
  Description of Artificial Sequence Synthetic peptide
GFTFSSYGMH

SEQ ID NO: 12 - protein, 15 aa, Artificial Sequence
  Description of Artificial Sequence Synthetic peptide
VIWYDGSNKYYADSV

SEQ ID NO: 13 - protein, 24 aa, Artificial Sequence
  Description of Artificial Sequence Synthetic peptide
TAVYYCARDPRGATLYYYYYGMDV

SEQ ID NO: 14 - protein, 11 aa, Artificial Sequence
  Description of Artificial Sequence Synthetic peptide
RASQSINSYLD

SEQ ID NO: 15 - protein, 7 aa, Artificial Sequence
  Description of Artificial Sequence Synthetic peptide
AASSLQS

SEQ ID NO: 16 - protein, 9 aa, Artificial Sequence
  Description of Artificial Sequence Synthetic peptide
QQYYSTPFT

(19) United States
(12) Patent Application Publication
(10) Pub. No.: US 2019/0256603 A1
(43) Pub. Date: Aug. 22, 2019

(54) ANTI-PD-L1 AND ANTI-CTLA-4 ANTIBODIES FOR TREATING NON-SMALL CELL LUNG CANCER

(71) Applicants: MEDIMMUNE, LLC, GAITHERSBURG, MD (US); C/O DEFINIENS AG, MINICH (DE)

(72) Inventors: KEITH STEELE, GAITHERSBURG, MD (US); SONG WU, GAITHERSBURG, MD (US); BRANDON HIGGS, GAITHERSBURG, MD (US); MORITZ WIDMAIER, MUNICH (DE); SONJA ALTHAMMER, MINICH (DE); RENE KORN, MINICH (DE); ANDREAS SPITZMUELLER, MINICH (DE)

(21) Appl. No.: 16/346,268

(22) PCT Filed: Nov. 10, 2017
(86) PCT No.: PCT/US2017/061085

§ 371 Date: Apr. 30, 2019

Related U.S. Application Data
(60) Provisional application No. 62/420,735, filed on Nov. 11, 2016.

(51) Int. Cl. C07K 16/28; A61P 35/00; G01N 33/574

(52) U.S. Cl. CPC C07K 16/2827; C07K 16/2818; A61P 35/00; G01N 2333/70517; G01N 2333/70596; C07K 2317/76; C07K 2317/21; G01N 33/57423

(57) ABSTRACT

Provided herein are methods of treating non-small cell lung cancers comprising administering an effective amount of durvalumab (MEDI4736) or an antigen-binding fragment thereof and tremelimumab or an antigen-binding fragment thereof. A combination of durvalumab and tremelimumab was effective at treating non-small cell lung cancers characterized as PD-L1- and having a high level of CD8+ tumor-infiltrating lymphocytes.

### BACKGROUND

Cancer continues to be a major global health burden. Despite progress in the treatment of cancer, there continues to be an unmet medical need for more effective and less toxic therapies, especially for those patients with advanced disease or cancers that are resistant to existing therapeutics.

The role of the immune system, in particular T cell-mediated cytotoxicity, in tumor control is well recognized. There is mounting evidence that T cells control tumor growth and survival in cancer patients, both in early and late stages of the disease. However, tumor-specific T-cell responses are difficult to mount and sustain in cancer patients.

Two T cell pathways receiving significant attention to date signal through cytotoxic T lymphocyte antigen-4 (CTLA-4, CD152) and programmed death ligand 1 (PD-L1, also known as B7-H1 or CD274).

CTLA4 is expressed on activated T cells and serves as a co-inhibitor to keep T cell responses in check following CD28-mediated T cell activation. CTLA4 is believed to regulate the amplitude of the early activation of naïve and memory T cells following TCR engagement and to be part of a central inhibitory pathway that affects both antitumor immunity and autoimmunity. CTLA4 is expressed exclusively on T cells, and the expression of its ligands CD80 (B7.1) and CD86 (B7.2), is largely restricted to antigen-presenting cells, T cells, and other immune mediating cells. Antagonistic anti-CTLA4 antibodies that block the CTLA4 signaling pathway have been reported to enhance T cell activation. One such antibody, ipilimumab, was approved by the FDA in 2011 for the treatment of metastatic melanoma. Another anti-CTLA4 antibody, tremelimumab, was tested in phase III trials for the treatment of advanced melanoma, but did not significantly increase the overall survival of patients compared to the standard of care (temozolomide or dacarbazine) at that time.

PD-L1 is also part of a complex system of receptors and ligands that are involved in controlling T-cell activation. In normal tissue, PD-L1 is expressed on T cells, B cells, dendritic cells, macrophages, mesenchymal stem cells, bone marrow-derived mast cells, as well as various nonhematopoietic cells. Its normal function is to regulate the balance between T-cell activation and tolerance through interaction with its two receptors: programmed death 1 (also known as PD-1 or CD279) and CD80 (also known as B7-1 or B7.1). PD-L1 is also expressed by tumors and acts at multiple sites to help tumors evade detection and elimination by the host immune system. PD-L1 is expressed in a broad range of cancers with a high frequency. In some cancers, expression of PD-L1 has been associated with reduced survival and unfavorable prognosis. Antibodies that block the interaction between PD-L1 and its receptors are able to relieve PD-L1-dependent immunosuppressive effects and enhance the cytotoxic activity of antitumor T cells in vitro. Durvalumab (MEDI4736) is a human monoclonal antibody directed against human PD-L1 that is capable of blocking the binding of PD-L1 to both the PD-1 and CD80 receptors.

Improving survival of lung cancer patients remains difficult despite improved medical therapies. Methods of characterizing lung cancer are useful for stratifying patients, thereby quickly directing them to effective therapies. Improved methods for predicting the responsiveness of subjects having lung cancer are urgently required as are new compositions and methods for treating lung cancer.

### BRIEF SUMMARY

The invention provides a method of treating non-small cell lung cancer (NSCLC) in a human patient, involving administering durvalumab (MEDI4736), or an antigen-binding fragment thereof, and tremelimumab, or an antigen-binding fragment thereof a patient identified as having PD-L1⁻ non-small cell lung cancer (NSCLC) having a high level of CD8⁺ tumor-infiltrating lymphocytes.

In another aspect, the invention provides a method of identifying a patient as having NSCLC that is responsive to treatment with durvalumab, or an antigen-binding fragment thereof, and tremelimumab, or an antigen-binding fragment thereof, the method involving detecting a high level of CD8⁺ tumor-infiltrating lymphocytes in a biological sample (e.g., a tumor biopsy). In particular embodiments, the NSCLC is a PD-L1⁻ or PD-L1⁺ NSCLC.

In another aspect, the invention provides a pharmaceutical composition containing durvalumab, or an antigen-binding fragment thereof, and tremelimumab, or an antigen-binding fragment thereof, for the treatment of a patient identified as having PD-L1⁻ non-small cell lung cancer (NSCLC) having a high level of CD8⁺ tumor-infiltrating lymphocytes.

In various embodiments of any aspect delineated herein, the level of CD8⁺ tumor-infiltrating lymphocytes is greater than about 300-350 cells/mm². In particular embodiments, the level of CD8⁺ tumor-infiltrating lymphocytes is greater than about 300-325 cells/mm². In a specific embodiment, the level of CD8⁺ tumor-infiltrating lymphocytes is greater than about 317 cells/mm². In various embodiments, the level of CD8⁺ tumor-infiltrating lymphocytes is measured prior to treatment. In various embodiments, the level of CD8⁺ tumor-infiltrating lymphocytes is measured in a tumor biopsy (i.e., obtained from the patient).

In various embodiments of any aspect delineated herein, durvalumab (MEDI4736) or an antigen-binding fragment thereof is administered at a dose of about 1 mg/kg, 3 mg/kg, 10 mg/kg, 15 mg/kg, or 20 mg/kg to a patient identified as having a PD-L1⁻ or PD-L1⁺ NSCLC. In various embodiments of any aspect delineated herein, tremelimumab or an antigen-binding fragment thereof is administered at a dose of about 1 mg/kg, 3 mg/kg, 10 mg/kg) to a patient identified as having a PD-L1⁻ or PD-L1⁺ NSCLC. In certain embodiments, durvalumab is administered at 20 mg/kg and tremelimumab is administered at 1 mg/kg. In certain embodiments, durvalumab is administered at 20 mg/kg every 4 weeks and tremelimumab is administered at 1 mg/kg.

In various embodiments of any aspect delineated herein, durvalumab is administered every 4 weeks. In various embodiments of any aspect delineated herein, durvalumab is administered every 2 weeks.

In various embodiments of any aspect delineated herein, durvalumab, or an antigen-binding fragment thereof, and tremelimumab, or an antigen-binding fragment thereof, are administered concurrently.

The method of any one of claims 1-16, wherein the durvalumab, or antigen-binding fragment thereof, is administered by intravenous injection.

The method of any one of claims 1-16, wherein the tremelimumab, or antigen-binding fragment thereof, is administered by intravenous injection.

In various embodiments of any aspect delineated herein, the administration results in an increased tumor response, a decrease in tumor size, or increase in objective response rate as compared to the administration of administration of durvalumab, or an antigen-binding fragment thereof, alone. In certain embodiments, the administration reduces tumor size by at least about 10%, 15%, 20%, 25%, 30%, 40%, 50%, 60%, 75%, 80%90% or more, including up to 100%, relative to baseline.

In various embodiments of any aspect delineated herein, the administration of durvalumab or an antigen-binding fragment thereof is by intravenous infusion. In various embodiments of any aspect delineated herein, the administration of tremelimumab or an antigen-binding fragment thereof is by intravenous infusion.

### BRIEF DESCRIPTION OF THE DRAWINGS/FIGURES

FIGS. 1A and 1B depict modified zone-based design for intermediate dosing.

FIG. 1A is a schematic showing main Q4W dose-escalation schedule. FIG. 1B is a schematic showing alternative Q2W dose-escalation schedule.

FIG. 2 is a graph depicting time to RECIST response (confirmed and unconfirmed) and duration of response. PD=progressive disease; PD-L1=programmed cell death ligand-1; RECIST=Response Evaluation Criteria In Solid Tumors.

FIGS. 3A-3C depict change from baseline in tumor size (response evaluable population with ≥24 weeks follow-up). FIG. 3A is a spider plot depicting change in tumor size from baseline for the combined T1 cohort. FIG. 3B is a spider plot depicting change in tumor size from baseline for the combined T3 cohort. FIG. 3C is a spider plot depicting change in tumor size from baseline for the T10 cohort.

FIGS. 4A-4D depict antitumor activity according to PD-L1 status (response evaluable population with ≥24 weeks follow-up). FIG. 4A is a spider plot depicting change in tumor size from baseline in PD-L1⁻ patients. FIG. 4B is a spider plot depicting change in tumor size from baseline in PD-L1⁺ patients. FIG. 4C is a spider plot depicting change in tumor size from baseline in patients with unknown PD-L1 status. FIG. 4D is a spider plot depicting best change in tumor size by PD-L1 status. D=durvalumab; na=status unknown; PD-L1=programmed cell death ligand-1; Q=every; T=tremelimumab; W=weeks.

FIGS. 5A-5D depict antitumor activity according to CD8+ status in PD-L1⁻ and PD-L1⁺ patients treated with durvalumab and tremelimumab in combination or with durvalumab monotherapy. FIG. 5A is a plot depicting best percentage change from baseline in target lesions (based on investigator assessment) by pretreatment CD8⁺ status (high/low CD8⁺ defined as above/below median density of CD8⁺ lymphocytes, i.e. 317 cells/mm²) in baseline tumor biopsies determined to be PD-L1 negative or positive and also in the context of treatment with durvalumab combined with tremelimumab versus monotherapy (cohort of NSCLC patients treated with durvalumab 10 mg/kg q2w)⁸. Error bars depict standard error of the mean (SEM). Patients receiving combination therapy with biopsies that were PD-L1⁻ but had high CD8⁺ tumor-infiltrating lymphocyte (TIL) levels at baseline showed a tendency towards greater tumor shrinkage compared to those treated with durvalumab alone (top panel). Conversely, patients with low CD8⁺ lymphocyte levels had a tendency towards tumor size changes of similar degree between the two treatment groups. In those with PD-L1⁺ tumors with high CD8+ lymphocyte levels at baseline, there was no obvious difference in tumor size change between treatment groups (bottom panel). FIGS. 5B-5D depict visualization of tissue from a CD8+ TIL high/PD-L1⁻ patient. FIG. 5B depicts CD8 IHC (naked image) of tissue from the CD8⁺ TIL high/PD-L1⁻ patient. FIG. 5C depicts Definiens classification of the image in FIG. 5B. FIG. 5D is an image of tissue from PD-L1⁻ tumor.

FIG. 6 is a graph depicting suppression of serum free sPD-L1 observed in patients treated with durvalumab and tremelimumab in combination (n=69). Two patients (D10 q4w/T1, PD due to non-target lesion from first disease assessment, treated after PD; D15 q4w/T1, unconfirmed response and treated after PD) showed partial free sPD-L1 suppression at some visits followed by complete suppression after repeated dosing. One patient (D15 q4w/T10, with one disease assessment and best overall response of PD) who was ADA positive with an impact on PK showed partial free sPD-L1 suppression on Day 29.

FIGS. 7A-7D depict T-cell proliferation and activation by flow cytometry. In FIGS. 7A-7C, all durvalumab doses are combined, with durvalumab monotherapy data⁸ shown in comparison. FIG. 7A is a graph showing percent change from baseline of CD4+ Ki67⁺ proliferating cells. FIG. 7B is a graph showing percent change from baseline of CD8⁺ Ki67⁺ proliferating cells. FIG. 7C is a graph showing percent change from baseline of CD4⁺ HLR-DR+ proliferating cells. FIG. 7D is a graph showing percent change from baseline of CD4⁺ Ki67⁺ proliferating cells by durvalumab dose, at 1 mg/kg tremelimumab.

FIGS. 8A and 8B depict serum concentrations of durvalumab and tremelimumab in combination. FIG. 8A is a graph depicting durvalumab serum concentrations. FIG. 8B is a graph depicting tremelimumab serum concentrations.

### DETAILED DESCRIPTION

It is to be noted that the term “a” or “an” entity refers to one or more of that entity; for example, “an antibody” is understood to represent one or more antibodies. As such, the terms “a” (or “an”), “one or more,” and “at least one” can be used interchangeably herein.

Provided herein are methods for treating non-small cell lung cancer (NSCLC) using durvalumab (MEDI4736) and tremelimumab and for identifying an NSCLC as responsive to treatment with durvalumab and tremelimumab. As described herein, it has been found that a combination of durvalumab and tremelimumab are effective at treating non-small cell lung cancers characterized as PD-L1⁻ and having a high level of CD8⁺ tumor-infiltrating lymphocytes. The invention is based at least in part on these discoveries. The methods provided include administering an effective amount of durvalumab or an antigen-binding fragment thereof and tremelimumab or an antigen-binding fragment thereof to treat PD-L1⁻ non-small cell lung cancer (NSCLC) having a high level of CM⁺ tumor-infiltrating lymphocytes (e.g., 300-350 cells/mm²).

There are three main subtypes of NSCLC: squamous cell carcinoma, adenocarcinoma, and large cell (undifferentiated) carcinoma. Other subtypes include adenosquamous carcinoma and sarcomatoid carcinoma. NSCLC may comprise a mutation in KRAS or in the Epidermal Growth Factor receptor. Such mutations are known in the art and described, for example, by Riely et al., Proc Am Thorac Soc. 2009 Apr 15; 6(2):201-5, which is incorporated herein by reference.

The combination of programmed cell death-1/programmed cell death ligand-1 (PD-1/PD-L1) pathway and cytotoxic T-lymphocyte-associated antigen-4 (CTLA-4) pathway blockade targets two compartments: anti-PD-L1/anti-PD-1 acts in the tumor microenvironment and blocks inhibition of T-cell function, whereas anti-CTLA-4 acts in the lymphoid compartment to expand the number and repertoire of tumor-reactive T cells.^{1.2} In a study of nivolumab (1 mg/kg every 3 weeks) plus ipilimumab (3 mg/kg every 3 weeks) for melanoma, progression-free survival with the combination was equivalent or greater than with either agent alone in both PD-L1-positive (PD-L1⁺) and PD-L1-negative (PD-L1⁻) tumors.³ However, a higher percentage of patients experienced treatment-related Grade 3/4 adverse events (AEs) with the combination compared with those receiving either agent alone. In addition, the same dose and schedule did not appear to be tolerated in NSCLC⁴, highlighting the need for optimal dose selection in this population to minimize the toxicity of combination regimens while maintaining clinical activity.

Durvalumab (MEDI4736) is a selective, high-affinity human IgG1 monoclonal antibody (mAb) that blocks PD-L1 binding to PD-1 and CD80⁴ but does not bind to programmed-cell death (PD-L2),⁵ avoiding potential immune-related toxicity due to PD-L2 blockade that is observed in susceptible animal models.^{6.7} In an ongoing Phase 1/2 study, durvalumab monotherapy has produced durable responses in patients with advanced NSCLC, with a manageable tolerability profile; confirmed/unconfirmed ORR with durvalumab 10 mg/kg every 2 weeks (q2w) was 27% in PD-L1⁺ patients, and 5% in PD-L1⁻ patients.⁸ In that study, a maximum tolerated dose (MTD) was not reached in the dose-escalation phase, and dose-expansion cohorts were initiated using a dose of 10 mg/kg q2w.⁸ Tremelimumab (CP-675,206) is a selective human IgG2 mAb inhibitor of CTLA-4⁹; it promotes T cell activity through CTLA-4 inhibition, but does not appear to directly deplete regulatory T cells.¹⁰ The combination of durvalumab and tremelimumab was based on strong preclinical data indicating that the two pathways are non-redundant, which suggests that targeting both may have additive or synergistic effects.¹¹ The results of the dose-escalation part of a Phase 1b study are described herein evaluating the tolerability and antitumor activity of this combination in patients with advanced NSCLC, regardless of PD-L1 expression status.

By “Durvalumab” (also known as “MEDI4736”) is meant an antibody or antigen binding fragment thereof that selectively binds a PD-L1 polypeptide and comprises at least a portion of a light chain variable region comprising the amino acid sequence of SEQ ID NO:1 and/or at least a portion of a heavy chain variable region comprising the amino acid sequence of SEQ ID NO:2.

Information regarding durvalumab (or antigen-binding fragments thereof) for use in the methods provided herein can be found in U.S. Pat. No. 8,779,108, the disclosure of which is incorporated herein by reference in its entirety. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (ADCC). Durvalumab is selective for PD-L1 and blocks the binding of PD-L1 to the PD-1 and CD80 receptors. Durvalumab can relieve PD-L1-mediated suppression of human T-cell activation in vitro and inhibits tumor growth in a xenograft model via a T-cell dependent mechanism.

Durvalumab for use in the methods provided herein comprises a heavy chain and a light chain or a heavy chain variable region and a light chain variable region. In a specific aspect, durvalumab or an antigen-binding fragment thereof for use in the methods provided herein comprises a light chain variable region comprising the amino acid sequence of SEQ ID NO:1 and a heavy chain variable region comprising the amino acid sequence of SEQ ID NO:2. In a specific aspect, durvalumab or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs:3-5, and wherein the light chain variable region comprises the Kabat-defined CDR1, CDR2, and CDR3 sequences of SEQ ID NOs:6-8. Those of ordinary skill in the art would easily be able to identify Chothia-defined, Abm-defined or other CDR definitions known to those of ordinary skill in the art. In a specific aspect, durvalumab or an antigen-binding fragment thereof for use in the methods provided herein comprises the variable heavy chain and variable light chain CDR sequences of the 2.14H9OPT antibody as disclosed in U.S. Pat. No. 8,779,108, which is herein incorporated by reference in its entirety.

By “Tremelimumab” is meant an antibody or antigen binding fragment thereof that selectively binds a CTLA4 polypeptide and comprises at least a portion of a light chain variable region comprising the amino acid sequence of SEQ ID NO:9 and/or at least a portion of a heavy chain variable region comprising the amino acid sequence of SEQ ID NO:10. Exemplary anti- CTLA4 antibodies are described for example at U.S. Pat. Nos. 6,682,736; 7,109,003; 7,123,281; 7,411,057; 7,824,679; 8,143,379; 7,807,797; and 8,491,895 (Tremelimumab is 11.2.1, therein), which are herein incorporated by reference. Tremelimumab is an exemplary anti-CTLA4 antibody. Tremelimumab sequences are provided in the sequence listing below.

Information regarding tremelimumab (or antigen-binding fragments thereof) for use in the methods provided herein can be found in U.S. Pat. No. 6,682,736 (where it is referred to as 11.2.1, the disclosure of which is incorporated herein by reference in its entirety. Tremelimumab (also known as CP-675,206, CP-675, CP-675206, and ticilimumab) is a human IgG₂ monoclonal antibody that is highly selective for CTLA4 and blocks binding of CTLA4 to CD80 (B7.1) and CD86 (B7.2). It has been shown to result in immune activation in vitro and some patients treated with tremelimumab have shown tumor regression.

Tremelimumab and antigen-binding fragments thereof for use in the methods provided herein comprises a heavy chain and a light chain or a heavy chain variable region and a light chain variable region. In a specific aspect, tremelimumab or an antigen-binding fragment thereof for use in the methods provided herein comprises a light chain variable region comprising the amino acid sequence of SEQ ID NO:9 and a heavy chain variable region comprising the amino acid sequence of SEQ ID NO:10. In a specific aspect, tremelimumab or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs:11-13, and wherein the light chain variable region comprises the Kabat-defined CDR1, CDR2, and CDR3 sequences of SEQ ID NOs:14-16. Those of ordinary skill in the art would easily be able to identify Chothia-defined, Abm-defined or other CDR definitions known to those of ordinary skill in the art. In a specific aspect, tremelimumab or an antigen-binding fragment thereof for use in the methods provided herein comprises or the variable heavy chain and variable light chain CDR sequences of the 11.2.1 antibody as disclosed in U.S. Pat. No. 6,682,736, which is herein incorporated by reference in its entirety.

The term “antigen binding fragment” refers to a portion of an intact antibody and/or refers to the antigenic determining variable regions of an intact antibody. It is known that the antigen binding function of an antibody can be performed by fragments of a full-length antibody. Examples of antibody fragments include, but are not limited to, Fab, Fab′, F(ab′)2, and Fv fragments, linear antibodies, single chain antibodies, diabodies, and multispecific antibodies formed from antibody fragments.

In certain aspects, a patient presenting with a NSCLC is administered durvalumab or an antigen-binding fragment thereof and tremelimumab or an antigen-binding fragment thereof. Durvalumab or an antigen-binding fragment thereof and tremelimumab or an antigen-binding fragment thereof can be administered only once or infrequently while still providing benefit to the patient. In further aspects the patient is administered additional follow-on doses. Follow-on doses can be administered at various time intervals depending on the patient's age, weight, clinical assessment, tumor burden, and/or other factors, including the judgment of the attending physician.

The intervals between doses of durvalumabor an antigen-binding fragment thereof can be every four weeks. The intervals between doses of tremelimumab or an antigen-binding fragment thereof can be every four weeks. The intervals between doses of tremelimumab or an antigen-binding fragment thereof can be every twelve weeks. The intervals between doses of tremelimumab or an antigen-binding fragment thereof can be every four weeks for six cycles and then every twelve weeks.

In certain aspects, durvalumab or an antigen-binding fragment thereof is administered about as frequently as tremelimumab or an antigen-binding fragment thereof. In certain aspects, durvalumab or an antigen-binding fragment thereof is administered about three times as frequently as tremelimumab or an antigen-binding fragment thereof.

In some embodiments, at least two doses of Durvalumab or an antigen-binding fragment thereof and tremelimumab or an antigen-binding fragment thereof are administered to the patient. In some embodiments, at least three doses, at least four doses, at least five doses, at least six doses, at least seven doses, at least eight doses, at least nine doses, at least ten doses, or at least fifteen doses or more can be administered to the patient. In some embodiments, Durvalumab or an antigen-binding fragment thereof is administered over a four-week treatment period, over an eight-week treatment period, over a sixteen-week treatment period, over a twenty-week treatment period, over a twenty-four-week treatment period, or over a one-year or more treatment period. In some embodiments, tremelimumab or an antigen-binding fragment thereof is administered over a four-week treatment period, over an eight-week treatment period, over a twelve-week treatment period, over a sixteen-week treatment period, over a twenty-week treatment period, over a twenty-four-week treatment period, over a thirty-six-week treatment period, over a forty-eight-week treatment period, or over a one-year or more treatment period.

In some embodiments, durvalumab or an antigen-binding fragment thereof and tremelimumab or an antigen-binding fragment thereof are administered on the same day. In some embodiments, durvalumab or an antigen-binding fragment thereof is administered at the same time as tremelimumab or an antigen-binding fragment thereof. In other embodiments, durvalumab or an antigen-binding fragment thereof is administered about 1 hour following administration of tremelimumab or an antigen-binding fragment thereof.

The amount of durvalumab or an antigen-binding fragment thereof and the amount of tremelimumab or an antigen-binding fragment thereof to be administered to the patient will depend on various parameters such as the patient's age, weight, clinical assessment, tumor burden and/or other factors, including the judgment of the attending physician.

In certain aspects the patient is administered one or more doses of durvalumab or an antigen-binding fragment thereof wherein the dose is about 1 mg/kg. In certain aspects the patient is administered one or more doses of durvalumab or an antigen-binding fragment thereof wherein the dose is about 3 mg/kg. In certain aspects the patient is administered one or more doses of durvalumab or an antigen-binding fragment thereof wherein the dose is about 10 mg/kg. In certain aspects the patient is administered one or more doses of durvalumab or an antigen-binding fragment thereof wherein the dose is about 15 mg/kg. In certain aspects the patient is administered one or more doses of durvalumab or an antigen-binding fragment thereof wherein the dose is about 20 mg/kg.

In certain aspects the patient is administered at least two doses of durvalumab or an antigen-binding fragment thereof wherein the dose is about 1 mg/kg. In certain aspects the patient is administered at least two doses of durvalumab or an antigen-binding fragment thereof wherein the dose is about 3 mg/kg. In certain aspects the patient is administered at least two doses of durvalumab or an antigen-binding fragment thereof wherein the dose is about 10 mg/kg. In certain aspects the patient is administered at least two doses of durvalumab or an antigen-binding fragment thereof wherein the dose is about 15 mg/kg. In certain aspects the patient is administered at least two doses of durvalumab or an antigen-binding fragment thereof wherein the dose is about 20 mg/kg. In some embodiments, the at least two doses are administered about four weeks apart.

In certain aspects the patient is administered at least three doses of durvalumab or an antigen-binding fragment thereof wherein the dose is about 1 mg/kg. In certain aspects the patient is administered at least three doses of durvalumab or an antigen-binding fragment thereof wherein the dose is about 3 mg/kg. In certain aspects the patient is administered at least three doses of durvalumab or an antigen-binding fragment thereof wherein the dose is about 10 mg/kg. In certain aspects the patient is administered at least three doses of durvalumab or an antigen-binding fragment thereof wherein the dose is about 15 mg/kg. In certain aspects the patient is administered at least three doses of durvalumab or an antigen-binding fragment thereof wherein the dose is about 20 mg/kg. In some embodiments, the at least three doses are administered about four weeks apart.

In certain aspects the patient is administered one or more doses of tremelimumab or an antigen-binding fragment thereof wherein the dose is about 1 mg/kg. In certain aspects the patient is administered one or more doses of tremelimumab or an antigen-binding fragment thereof wherein the dose is about 3 mg/kg. In certain aspects the patient is administered one or more doses of tremelimumab or an antigen-binding fragment thereof wherein the dose is about 10 mg/kg.

In certain aspects the patient is administered at least two doses of tremelimumab or an antigen-binding fragment thereof wherein the dose is about 1 mg/kg. In certain aspects the patient is administered at least two doses of tremelimumab or an antigen-binding fragment thereof wherein the dose is about 3 mg/kg. In certain aspects the patient is administered at least two doses of tremelimumab or an antigen-binding fragment thereof wherein the dose is about 10 mg/kg. In some embodiments, the at least two doses are administered about four weeks apart. In some embodiments, the at least two doses are administered about twelve weeks apart.

In certain aspects the patient is administered at least three doses of tremelimumab or an antigen-binding fragment thereof wherein the dose is about 1 mg/kg. In certain aspects the patient is administered at least three doses of tremelimumab or an antigen-binding fragment thereof wherein the dose is about 3 mg/kg. In certain aspects the patient is administered at least three doses of tremelimumab or an antigen-binding fragment thereof wherein the dose is about 10 mg/kg. In some embodiments, the at least three doses are administered about four weeks apart. In some embodiments, the at least three doses are administered about twelve weeks apart.

In certain aspects, administration of durvalumab or an antigen-binding fragment thereof and/or tremelimumab or an antigen-binding fragment according to the methods provided herein is through parenteral administration. For example, durvalumab or an antigen-binding fragment thereof and/or tremelimumab or an antigen-binding fragment can be administered by intravenous infusion or by subcutaneous injection. In some embodiments, the administration is by intravenous infusion.

In certain aspects, 1 mg/kg of durvalumab or an antigen-binding fragment thereof and 1 mg/kg of tremelimumab or an antigen-binding fragment thereof are administered to a patient. In certain aspects, 1 mg/kg of durvalumab or an antigen-binding fragment thereof and 3 mg/kg of tremelimumab or an antigen-binding fragment thereof are administered to a patient. In certain aspects, 1 mg/kg of durvalumab or an antigen-binding fragment thereof and 10 mg/kg of tremelimumab or an antigen-binding fragment thereof are administered to a patient.

In certain aspects, 3 mg/kg of durvalumab or an antigen-binding fragment thereof and 1 mg/kg of tremelimumab or an antigen-binding fragment thereof are administered to a patient. In certain aspects, 3 mg/kg of durvalumab or an antigen-binding fragment thereof and 3 mg/kg of tremelimumab or an antigen-binding fragment thereof are administered to a patient. In certain aspects, 3 mg/kg of durvalumab or an antigen-binding fragment thereof and 10 mg/kg of tremelimumab or an antigen-binding fragment thereof are administered to a patient.

In certain aspects, 10 mg/kg of durvalumab or an antigen-binding fragment thereof and 1 mg/kg of tremelimumab or an antigen-binding fragment thereof are administered to a patient. In certain aspects, 10 mg/kg of durvalumab or an antigen-binding fragment thereof and 3 mg/kg of tremelimumab or an antigen-binding fragment thereof are administered to a patient. In certain aspects, 10 mg/kg of durvalumab or an antigen-binding fragment thereof and 10 mg/kg of tremelimumab or an antigen-binding fragment thereof are administered to a patient.

In certain aspects, 15 mg/kg of durvalumab or an antigen-binding fragment thereof and 1 mg/kg of tremelimumab or an antigen-binding fragment thereof are administered to a patient. In certain aspects, 15 mg/kg of durvalumab or an antigen-binding fragment thereof and 3 mg/kg of tremelimumab or an antigen-binding fragment thereof are administered to a patient. In certain aspects, 15 mg/kg of durvalumab or an antigen-binding fragment thereof and 10 mg/kg of tremelimumab or an antigen-binding fragment thereof are administered to a patient.

In certain aspects, 20 mg/kg of durvalumab or an antigen-binding fragment thereof and 1 mg/kg of tremelimumab or an antigen-binding fragment thereof are administered to a patient. In certain aspects, 20 mg/kg of durvalumab or an antigen-binding fragment thereof and 3 mg/kg of tremelimumab or an antigen-binding fragment thereof are administered to a patient. In certain aspects, 20 mg/kg of durvalumab or an antigen-binding fragment thereof and 10 mg/kg of tremelimumab or an antigen-binding fragment thereof are administered to a patient.

The methods provided herein can decrease, retard or stabilize tumor growth. In some aspects the reduction or retardation can be statistically significant. A reduction in tumor growth can be measured by comparison to the growth of patient's tumor at baseline, against an expected tumor growth, against an expected tumor growth based on a large patient population, or against the tumor growth of a control population. In certain aspects, a tumor response is measured using the Response Evaluation Criteria in Solid Tumors (RECIST).

In certain aspects, a tumor response is detectable at week 8. In certain aspects, a tumor response is detectable at week 33. In certain aspects, a tumor response is detectable at week 50.

In certain aspects, a tumor response is detectable after administration of administration of two doses of durvalumab or an antigen-binding fragment thereof and two doses of tremelimumab or an antigen-binding fragment thereof. In certain aspects, a tumor response is detectable after administration of administration of eight doses of durvalumab or an antigen-binding fragment thereof and seven doses of tremelimumab or an antigen-binding fragment thereof. In certain aspects, a tumor response is detectable after administration of administration of thirteen doses of durvalumab or an antigen-binding fragment thereof and nine doses of tremelimumab or an antigen-binding fragment thereof.

In certain aspects “objective response” (regarding antitumor activity) is defined as confirmed complete or partial response (CR or PR). In certain aspects “disease control” at 24 weeks is defined as CR, PR, or stable disease (SD) duration of >24 weeks. The objective response rate (ORR) and disease control rate (DCR) at 24 weeks are estimated and 95% confidence intervals (CIs) are calculated using the exact binomial distribution.

In certain aspects, a patient achieves disease control (DC). Disease control can be a complete response (CR), partial response (PR), or stable disease (SD).

A “complete response” (CR), a “partial response” (PR), and “stable disease”

(SD) can be determined as defined in Table 1 below.

TABLE 1
Evaluation of Overall Response

| Target Lesions | Non-target lesions | New Lesions | Overall Response |
|---|---|---|---|
| Complete | Complete Response | No | Complete |
| Response | | | Response |
| No target lesion^{a} | Complete Response | No | Complete |
| | | | Response |
| Complete | Not evaluable^{b} | No | Partial Response |
| Response | | | |
| Complete | Non-complete | No | Partial Response |
| Response | response/ non- | | |
| | progressive disease | | |
| Partial Response | Non-progressive | No | Partial Response |
| | disease and not | | |
| | evaluable^{b} | | |
| Stable Disease | Non-progressive | No | Stable Disease |
| | disease and not | | |
| | evaluable^{b} | | |
| Not all evaluated | Non-progressive | No | Not evaluable |
| | disease | | |
| No target lesion^{a} | Not all evaluated | No | Not evaluable |
| No target lesion^{a} | Non-complete | No | Non-complete |
| | response/non- | | response/non- |
| | progressive disease | | progressive disease |
| Progressive | Any | Yes or No | Progressive |
| Disease | | | Disease |
| Any | Progressive Disease | Yes or No | Progressive |
| | | | Disease |
| Any | Any | Yes | Progressive |
| | | | Disease |
| No target lesion^{a} | Unequivocal | Yes or No | Progressive |
| | progressive disease | | Disease |
| No target lesion^{a} | Any | Yes | Progressive |
| | | | Disease |

^{a}Defined as no target lesions at baseline.
^{b}Not evaluable is defined as either when no or only a subset of lesion measurements are made at an assessment.

In certain aspects, administration of durvalumab or an antigen-binding fragment thereof can increase progression-free survival (PFS).

In certain aspects, administration of durvalumab or an antigen-binding fragment thereof can increase overall survival (OS).

In some embodiments, the patient has previously received treatment with at least one chemotherapeutic agent. In some embodiments, the patient has previously received treatment with at least two chemotherapeutic agents. The chemotherapeutic agent can be, for example, and without limitation, Vemurafenib, Erlotinib, Afatinib, Cetuximab, Carboplatin, Bevacizumab, Erlotinib, Gefitinib, and/or Pemetrexed.

In some embodiments, the NSCLC is refractory or resistant to at least one chemotherapeutic agent. In some embodiments, the tumor is refractory or resistant to at least two chemotherapeutic agents. The tumor can be refractory or resistant to one or more of, for example, and without limitation, Vemurafenib, Erlotinib, Afatinib, Cetuximab, Carboplatin, Bevacizumab, Erlotinib, Gefitinib, and/or Pemetrexed. In some embodiments, the NSCLC is negative for PD-L1. In some embodiments, the NSCLC is positive for PD-L1.

In some embodiments, the patient has an Eastern Cooperative Oncology Group (ECOG) (Oken M M, et al. Am. J. Clin. Oncol. 5: 649-55 (1982)) performance status of 0 or 1 prior to the administration of durvalumab or an antigen-binding fragment thereof and tremelimumab or an antigen-binding fragment thereof.

According to the methods provided herein, administration of durvalumab or an antigen-binding fragment thereof and tremelimumab or an antigen-binding fragment thereof can result in desirable pharmacokinetic parameters as shown in some early data. Total drug exposure can be estimated using the “area under the curve” (AUC). “AUC (tau)” refers to AUC from time 0 to time τ, the dosing interval, whereas “AUC (inf)” refers to the AUC until infinite time. The administration can produce AUC (tau) of about 600 to about 3,000 μg/mL*day of durvalumab or antigen-binding fragment thereof and about 250 to about 350 μg/mL*day of tremelimumab or antigen-binding fragment thereof. The administration can produce a maximum observed concentration (Cmax) of about 60 to about 300 μg/mL durvalumab or antigen-binding fragment thereof and of about 25 to about 35 μg/mL tremelimumab or antigen-binding fragment thereof. The administration can produce a C trough (minimum plasma drug concentration) of about 5 to about 40 μg/mL durvalumab or antigen-binding fragment thereof and about 4 to about 6 μg/mL tremelimumab or antigen-binding fragment thereof.

As provided herein, durvalumab or an antigen-binding fragment thereof can also decrease free (soluble) PD-L1 levels. Free (soluble) PD-L1 refers to PD-L1 that is not bound (e.g., by durvalumab). In some embodiments, PD-L1 levels are reduced by at least 65%. In some embodiments, PD-L1 levels are reduced by at least 80%. In some embodiments, PD-L1 levels are reduced by at least 90%. In some embodiments, PD-L1 levels are reduced by at least 95%. In some embodiments, PD-L1 levels are reduced by at least 99%. In some embodiments, PD-L1 levels are not detectable following administration of durvalumab or an antigen-binding fragment thereof and tremelimumab or an antigen-binding fragment thereof.

In some embodiments, PD-L1 levels are reduced by at least 65% after a single administration of durvalumab or an antigen-binding fragment thereof. In some embodiments, PD-L1 levels are reduced by at least 80% after a single administration of durvalumab or an antigen-binding fragment thereof. In some embodiments, PD-L1 levels are reduced by at least 90% after a single administration of durvalumab or an antigen-binding fragment thereof. In some embodiments, PD-L1 levels are reduced by at least 95% after a single administration of durvalumab or an antigen-binding fragment thereof. In some embodiments, PD-L1 levels are reduced by at least 99% after a single administration of durvalumab or an antigen-binding fragment thereof. In some embodiments, PD-L1 levels are not detectable following a single administration of durvalumab or an antigen-binding fragment thereof.

In some embodiments, PD-L1 levels are reduced by at least 65% after administration of two doses of durvalumab or an antigen-binding fragment thereof. In some embodiments, PD-L1 levels are reduced by at least 80% after administration of two doses of durvalumab or an antigen-binding fragment thereof. In some embodiments, PD-L1 levels are reduced by at least 90% after administration of two doses of durvalumab or an antigen-binding fragment thereof. In some embodiments, PD-L1 levels are reduced by at least 95% after administration of two doses of durvalumab or an antigen-binding fragment thereof. In some embodiments, PD-L1 levels are reduced by at least 99% after administration of two doses of durvalumab or an antigen-binding fragment thereof. In some embodiments, PD-L1 levels are not detectable following administration of two doses of durvalumab or an antigen-binding fragment thereof.

Treatment of a patient with a solid tumor using both durvalumab or an antigen-binding fragment thereof and tremelimumab or an antigen-binding fragment thereof (i.e., co-therapy) as provided herein can result in an synergistic effect. As used herein, the term “synergistic” refers to a combination of therapies (e.g., a combination of durvalumab or an antigen-binding fragment thereof and tremelimumab or an antigen-binding fragment thereof) which is more effective than the additive effects of the single therapies.

A synergistic effect of a combination of therapies (e.g., a combination of a durvalumab or an antigen-binding fragment thereof and tremelimumab or an antigen-binding fragment thereof) permits the use of lower dosages of one or more of the therapeutic agents and/or less frequent administration of said therapeutic agents to a patient with a solid tumor. The ability to utilize lower dosages of therapeutic agents and/or to administer said therapies less frequently reduces the toxicity associated with the administration of said therapies to a subject without reducing the efficacy of said therapies in the treatment of a solid tumor. In addition, a synergistic effect can result in improved efficacy of therapeutic agents in the management, treatment, or amelioration of an solid tumor. The synergistic effect of a combination of therapeutic agents can avoid or reduce adverse or unwanted side effects associated with the use of either single therapy.

In co-therapy, durvalumab or an antigen-binding fragment thereof can be optionally included in the same pharmaceutical composition as the tremelimumab or an antigen-binding fragment thereof, or may be included in a separate pharmaceutical composition. In this latter case, the pharmaceutical composition comprising durvalumab or an antigen-binding fragment thereof is suitable for administration prior to, simultaneously with, or following administration of the pharmaceutical composition comprising tremelimumab or an antigen-binding fragment thereof. In certain instances, the durvalumab or an antigen-binding fragment thereof is administered at overlapping times as tremelimumab or an antigen-binding fragment thereof in a separate composition.

Subjects suffering from lung cancer (e.g., non-small cell lung cancer) may be tested for PD-L1 polynucleotide or polypeptide expression in the course of selecting a treatment method. Patients identified as having tumors that are negative for PD-L1 (e.g., as defined by Ct or IHC-M score) or by having reduced or undetectable levels of PD-L1 relative to a reference level are identified as responsive to treatment with a combination of an anti-PD-L1 antibody and tremelimumab. Such patients are administered an durvalumab, or an antigen-binding fragment thereof in combination with tremelimumab or an antigen-binding fragment thereof.

### EXAMPLES

### EXAMPLE 1: A study Evaluating Treatment with a Combination of Durvalumab and Tremelimumab in Patients with Advanced Non-Small Cell Lung Cancer

Programmed cell death ligand-1 (PD-L1) and cytotoxic T-lymphocyte-associated antigen-4 (CTLA-4) immune checkpoints inhibit antitumor T cell activity. Combining the anti-PD-L1 antibody durvalumab (MEDI4736) and the anti-CTLA-4 antibody tremelimumab may provide greater antitumor activity than monotherapy in patients with PD-L1-negative tumors. The results of an ongoing, multicenter, non-randomized, open-label Phase 1b study are described herein. This study evaluated durvalumab plus tremelimumab in patients with advanced non-small cell lung cancer. This study is the first to investigate the safety and antitumor activity of durvalumab in combination with the CTLA-4 inhibitor tremelimumab in previously treated patients with locally advanced or metastatic NSCLC.

Immunotherapy-naïve patients with confirmed locally advanced or metastatic

NSCLC were eligible for the study. Eligible patients were aged ≥18 years and had confirmed locally advanced or metastatic squamous or non-squamous NSCLC with one or more measurable lesions based on Response Evaluation Criteria in Solid Tumors (RECIST) guidelines 1.113, which is herein incorporated by reference in its entirety. In particular, patients were eligible regardless of PD-L1 expression (evaluated using an immunohistochemistry assay).

Patients had to be immunotherapy-naïve (with the exception of prior vaccines) but may have received any number of other systemic therapies. Patients entering the dose-escalation phase had not responded to, relapsed after, were unable to tolerate, or were not eligible for standard treatment. Other inclusion criteria included an Eastern Cooperative Oncology Group (ECOG) performance status (PS) 0-1 and adequate organ and marrow function. Patients with CNS metastases were required to be asymptomatic without concurrent treatment and to have had ≥28 days of non-progression of CNS metastases (except for those with leptomeningeal disease or cord compression, who were excluded). Study exclusion criteria included concurrent anticancer therapy (except localized palliative treatment); any investigational anticancer therapy ≥28 days before first doses of study drugs; prior severe or persistent immune-related adverse events (AEs); persistent AEs from prior anticancer therapy (except those judged unlikely to be exacerbated by study drugs); current or prior use (≥14 days before first doses of study drugs) of immunosuppressive medication (except intranasal/inhaled corticosteroids or systemic corticosteroids ≤10 mg prednisone equivalent); history of primary immunodeficiency; and human immunodeficiency virus or hepatitis A, B or C.

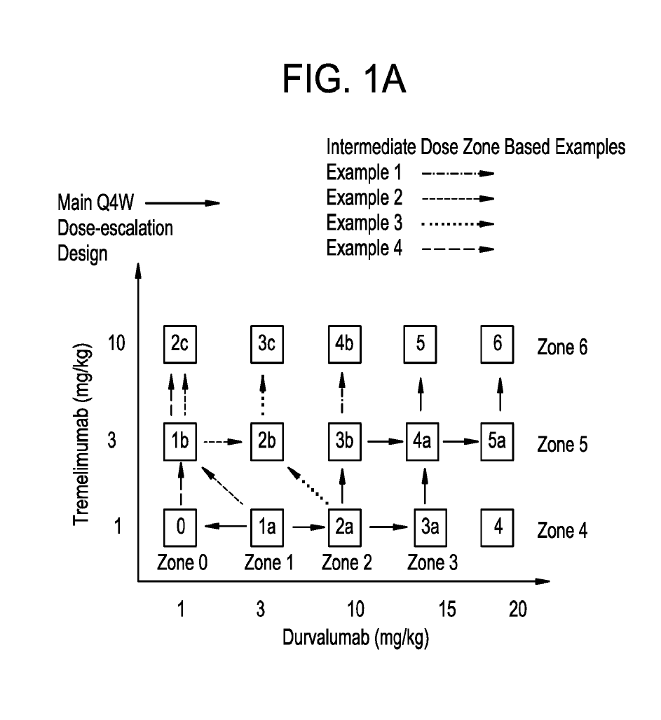

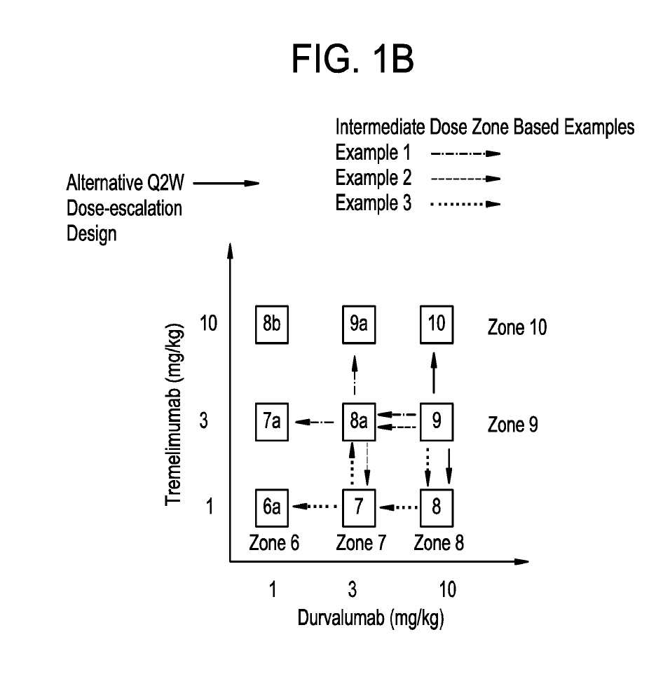

Dose combinations were zoned. In general, the modified zone-based design (FIGS. 1A and 1B) allows for the exploration of cohorts (comparison of multiple combinations of doses) in lower zones or within a zone. Exploration of higher zones can occur if a lower zone is used as an intermediate. If no more than ⅙ patients experienced a DLT in a given dose cohort, then dose-escalation continued until reaching the MTD or the highest protocol-defined dose for each agent. If the MTD is exceeded for 2 or more cohorts within a zone or for the starting dose cohort for 2 adjacent zones then further exploration to higher zones cannot occur even if a lower intermediate zone is evaluated.

A DLT was defined as any Grade 3 or higher drug-related toxicity that occurred from the first dose until administration of: (i) the third dose of durvalumab+tremelimumab (for the cohort receiving D3 q4w/T1); (ii) the second dose of durvalumab+tremelimumab (for all other cohorts receiving durvalumab q4w); or (iii) the third dose of durvalumab and second dose of tremelimumab (for cohorts receiving durvalumab q2w).

MTD evaluation was based on the dose-limiting toxicity (DLT) evaluable population (received protocol-assigned treatment and completed the DLT evaluation period or experienced a DLT during the DLT evaluation period). Non-evaluable patients in the dose-escalation phase could be replaced. Tolerability was based on the as-treated population (all patients receiving any dose of either study drug). Antitumor activity was based on the response evaluable population dosed ≥24 weeks prior to data cutoff. The response evaluable population included treated patients with measurable disease at baseline who had ≥1 follow-up scan or discontinued treatment due to disease progression or death without any follow-up scan. The median for duration of response is calculated based on the Kaplan-Meier method.

For antitumor activity, objective response was defined as confirmed complete or partial response (CR or PR), and disease control at 24 weeks was defined as CR, PR, or stable disease (SD) duration of ≥24 weeks. The objective response rate (ORR) and disease control rate (DCR) at 24 weeks were estimated and 95% confidence intervals (CIs) were calculated using the exact binomial distribution.

Safety and antitumor activity measures were evaluated by cohort and by combined cohorts for T1 and T3. The combined T1 cohort included all T1 cohorts except the D3 q4w/T1 cohort (n=3), as this was associated with low PK exposure and was considered to be a sub-therapeutic dose.

Study drugs were administered intravenously every four weeks (q4w) for 13 doses of durvalumab (D), and q4w for six doses followed by every 12 weeks (q12w) for three doses of tremelimumab (T). Patients were enrolled according to a standard 3+3 and modified zone-based design¹² (FIGS. 1A and 1B), with further expansion of escalation cohorts to allow for safety assessment. Multiple combinations of durvalumab 3 mg/kg (D3) to 20 mg/kg (D20) and tremelimumab 1 mg/kg (T1) to 3 mg/kg (T3) were explored (Table 2).

In particular, durvalumab doses of 3, 10, 15, or 20 mg/kg every 4 weeks (q4w) or 10 mg/kg q2w were combined with tremelimumab 1, 3, or 10 mg/kg q4w for six doses then q12w for three doses, including for example a D15 q4w/T10 combination. During the escalation phase, D10 q2w was also tested in combination with T1 or T3.

Study treatment was for 12 months or until progressive disease, DLT or other unacceptable toxicity, withdrawn consent, or discontinuation for other reasons. Patients who achieved and maintained disease control (i.e., complete response [CR], partial response [PR], or stable disease [SD]) through to the end of the 12-month treatment period entered follow-up. One round of re-treatment was offered if progressive disease was noted during follow-up and the patient had not received other treatments for their disease and still met the study eligibility criteria.

The primary endpoint of the dose-escalation phase was the safety of durvalumab in combination with tremelimumab (as determined by the MTD or the highest protocol-defined dose in the absence of exceeding the MTD) and the tolerability of the combination. AEs, serious AEs (SAEs), and laboratory abnormalities were classified and graded according to National Cancer Institute Common Terminology Criteria for AEs version 4.03 (NCI CTCAE v4.03) and monitored from the start of the study until 90 days after the last dose of study drugs. SAEs occurring ≥90 days post-last dose considered related to study treatment according to the investigator were also reported.

Secondary endpoints included antitumor activity, pharmacokinetic (PK) parameters (durvalumab and tremelimumab concentrations in serum), and immunogenicity (anti-drug antibodies [ADA]) measured with validated assays (Supplementary Appendix). Assessment of antitumor activity included investigator-reported response based on Response Evaluation Criteria In Solid Tumors (RECIST) version 1.1.¹³

Exploratory endpoints included pharmacodynamics parameters (free soluble PD-L 1 [sPD-L1] suppression and biomarkers assessing the biological activity of durvalumab in combination with tremelimumab). Target engagement for durvalumab was assessed using suppression of free soluble PD-L1 in serum (sPD-L1). sPD-L1 that is not bound by durvalumab was quantified using a validated electrochemiluminescence (ECL) method. Archival tumor or fresh tumor biopsies performed at baseline were assessed for PD-L1 and CD8 expression. PD-L1 immunohistochemical (IHC) staining of formalin-fixed, paraffin-embedded samples was performed on an automated BenchMark ULTRA® platform using the Ventana PD-L1 SP263 rabbit mAb assay.¹⁴ Clinical validation was done based on the durvalumab monotherapy study in NSCLC patients.⁸ Samples were considered positive if ≥25% of tumor cells demonstrated membrane staining for PD-L1 at any intensity. Automated scoring of CD8⁺ lymphocytes used Definiens Developer XD 2.1.4 software applied to digitized IHC slides.

Target engagement for durvalumab was assessed using suppression of free soluble PD-L1 in serum (sPD-L1). sPD-L1 that is not bound by durvalumab was quantified using a validated ECL method. Briefly, sPD-L1 was captured by biotinylated anti-PD-L1 antibody clone 2.7A4 (MedImmune) that competes with durvalumab for PD-L1 binding, and detected by anti-PD-L1 antibody clone 130021 (R&D Systems) plus ruthenium-labeled goat anti-mouse IgG. The ECL signal was measured by a Sector Imager 2400 or 6000 (MSD) and was proportional to serum concentration of sPD-L1. Serum sPD-L1 concentration was quantified by interpolating from sPD-L1 standard curves. T-cell proliferation and activation were assessed by flow cytometry; data for durvalumab monotherapy were reported previously.⁸ Pharmacodynamic data were summarized using descriptive and graphical approaches in Phoenix WinNonlin (Certara) and Prism (version 6.03 GraphPad Software).

The planned number of patients was dependent upon the toxicities observed as the study progressed, with the potential for up to approximately 118 evaluable patients (78 in q4w and 40 in q2w) to be enrolled.

### EXAMPLE 2: Results Show a Combination of Durvalumab and Tremelimumab is Effective for Treating Patients with Advanced Non-Small Cell Lung Cancer, Including PD-L1-/High CD8+ Patients.

102 patients were recruited into the dose-escalation phase of the study at five centers in the United States between Oct. 28, 2013 and Apr. 1, 2015. As of the Jun. 1, 2015 cutoff, all 102 patients had received study treatment in the dose-escalation phase and were included in the as-treated population.

Across all dose cohorts, median patient follow-up was 18.8 weeks (range 2-68). Patients received a median of 3 doses of durvalumab (range 1-13), and 3 doses (range 1-9) of tremelimumab. At the time of data cutoff, 4 patients had completed 1 year of treatment and were in follow-up, and twenty-six patients (25%) were still on treatment. Common reasons for discontinuation were AEs (26%), progressive disease (21%), and death (15%).

Mean age was 65.3 years (range 22-86), 54% of patients were male, 90% had non-squamous NSCLC, and 70% had an Eastern Cooperative Oncology Group performance status of 1; 39% had received 1 prior line of systemic therapy and 55% had received ≥2 prior lines (Table 2).

### (a) Antitumor Activity

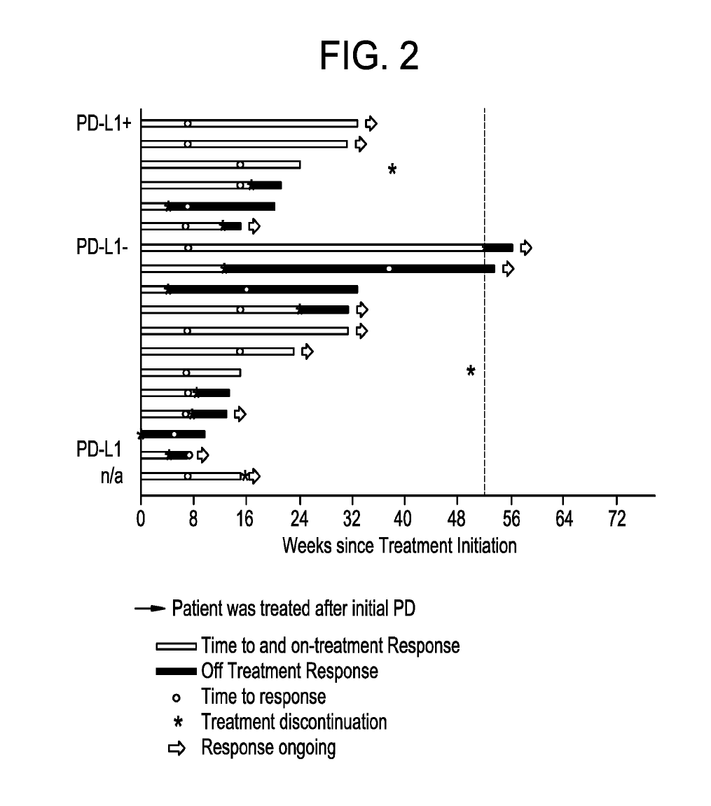

Across all cohorts, 63 patients were evaluable (≥24 weeks of follow-up). The ORR was 18% (95% CI, 9-29) and the DCR at 24 weeks was 29% (95% CI, 18-41) (Tables 3 and 4). Among the 11 patients with confirmed objective response, median time to response was 7.1 weeks (range, 6.7-15.9) and median duration of response was not reached (range, 6.1+-49.1+ weeks) (FIG. 2); response was ongoing in 9 patients at the time of data cutoff. Of the 4 patients who had completed 1 year of treatment and entered follow-up, 3 had progressive disease and 1 had 3 months of follow-up. In patients with PD-L1⁻ tumors (including those with no tumor cell membrane PD-L1 staining), ORR was 16% (95% CI, 6-32), and in patients with PD-L1⁺ tumors ORR was 22% (95% CI, 6-48). In the epidermal growth factor receptor/anaplastic lymphoma kinase wild-type population (n=58), the ORR was 19% (95% CI, 10-31).

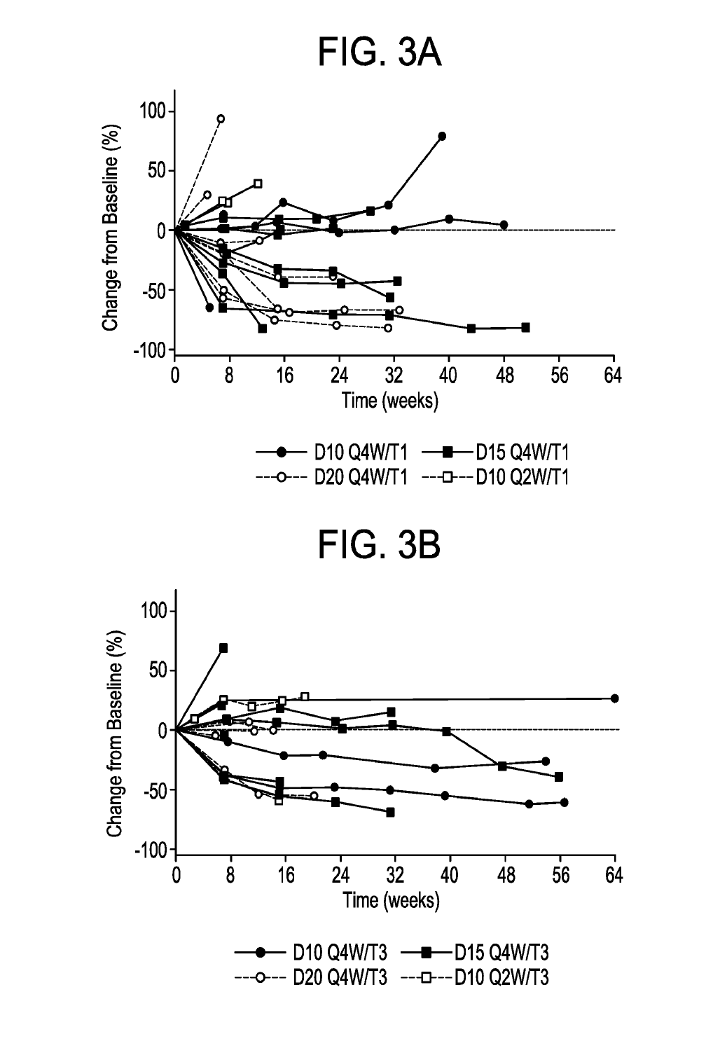

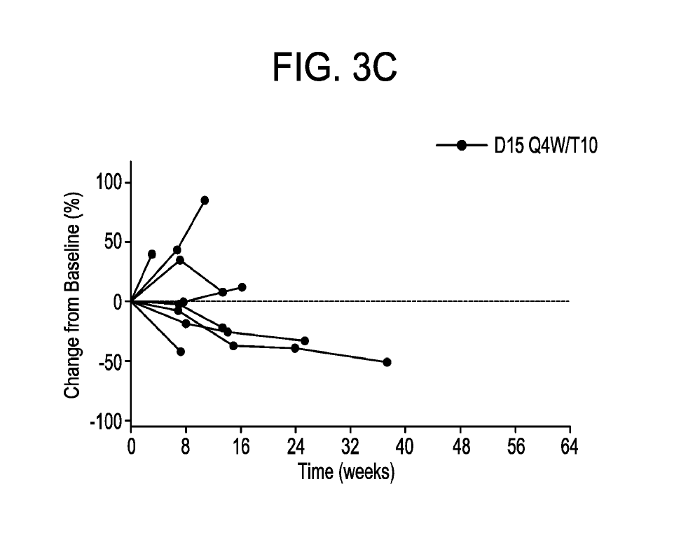

There were no responses in the lowest dose cohort (D3 q4w/T1, n=3), with progression on first scan among all patients. ORR was 23% (95% CI, 9-44) in the combined T1 cohort (n=26) and 38% (95% CI, 9-76) in the D20 q4w/T1 cohort (n=18). Higher doses of tremelimumab were not associated with higher response rates. Changes from baseline in tumor size in the combined T1 cohort, the combined T3 cohort, and the T10 cohort are shown in FIGS. 3A-3C.

(b) Antitumor Activity by PD-L1 and CD8 status

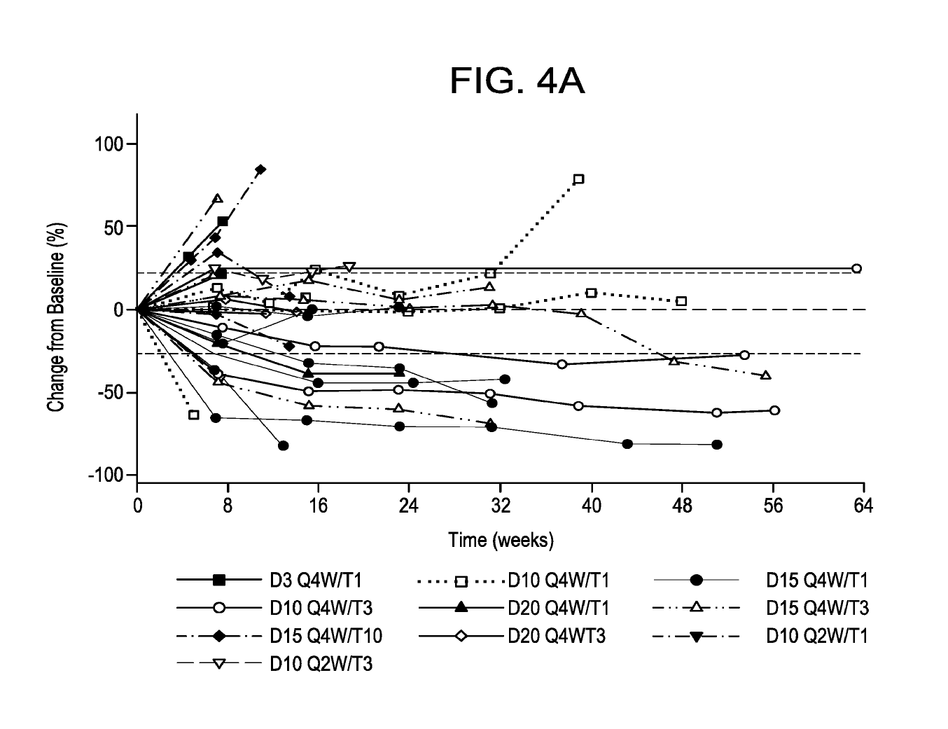

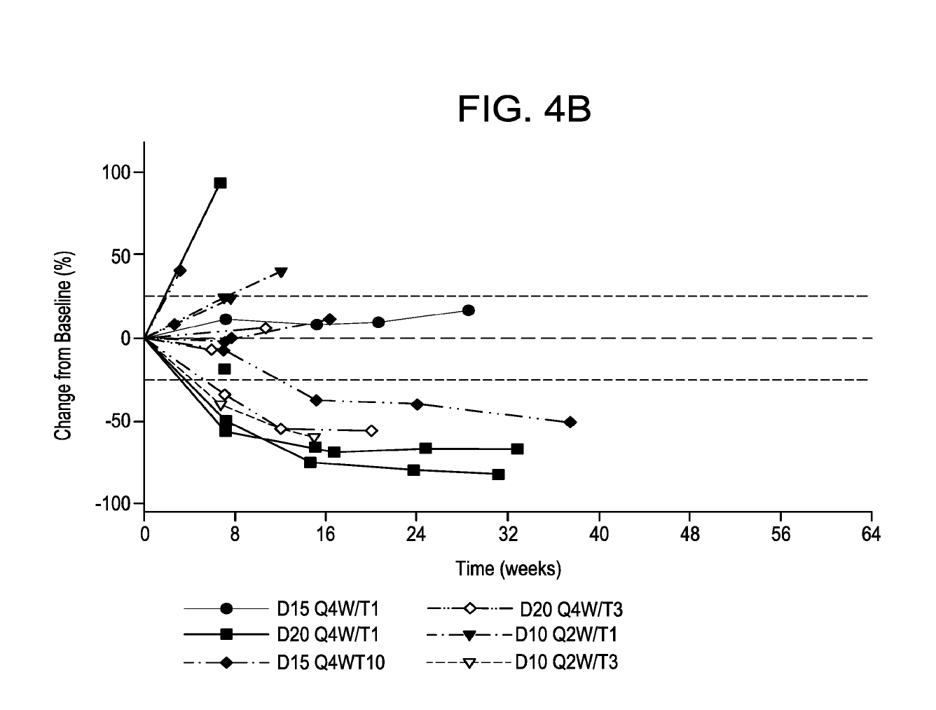

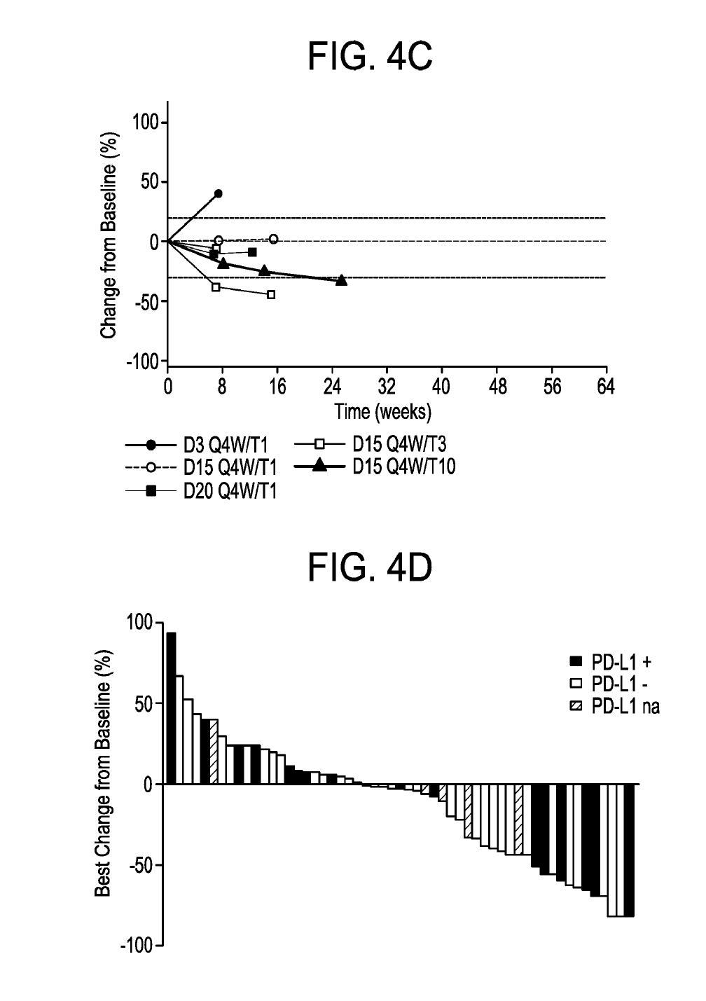

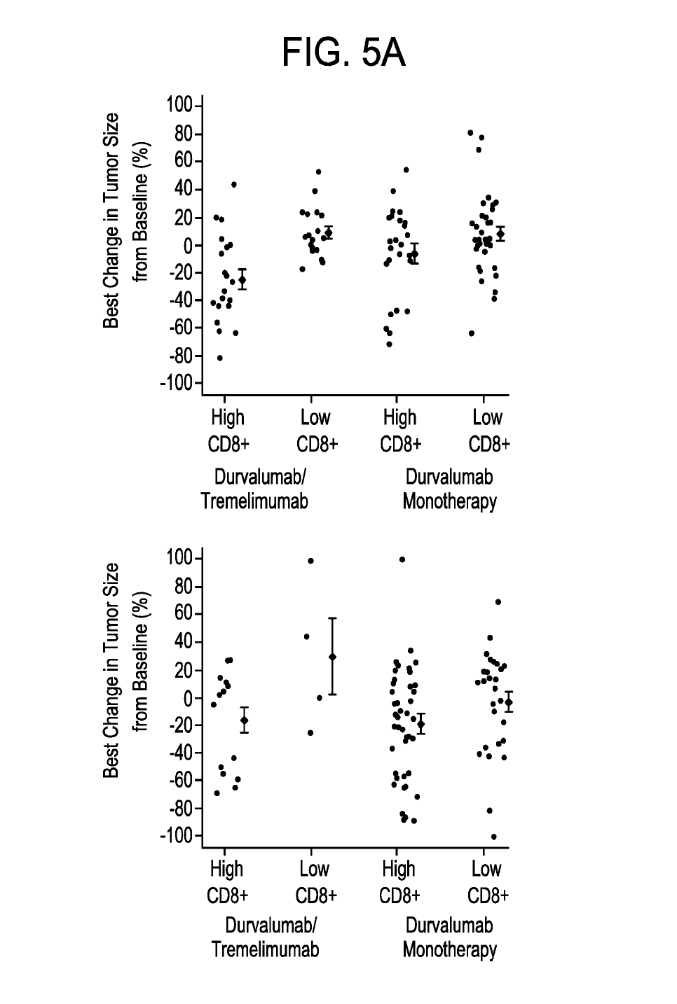

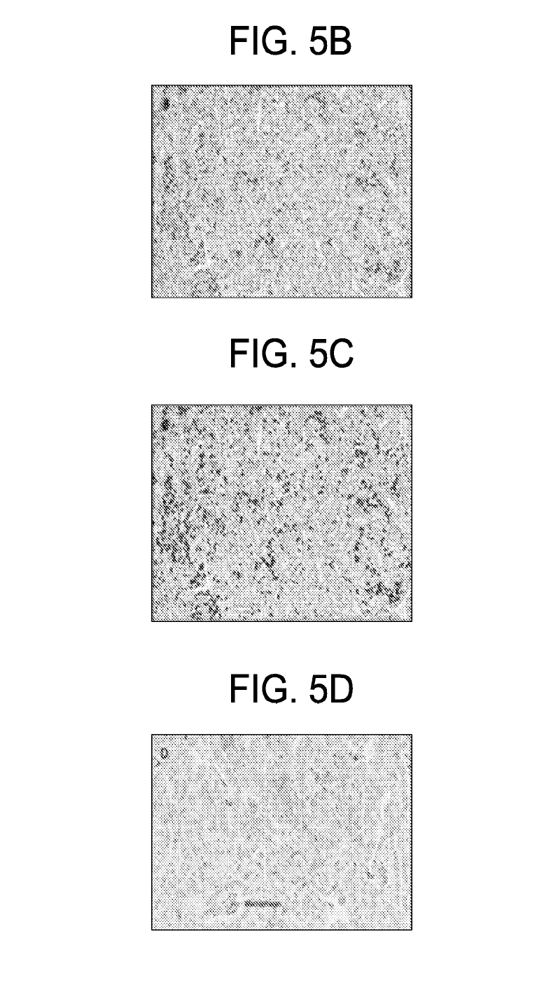

Antitumor activity was observed in patients with both PD-L1⁻ and PD-L1⁺ tumors, and few differences were noted among dosing cohorts (FIGS. 4A-4D; Tables 3 and 4). In an exploratory analysis of patients with ≥16 weeks follow-up, of 20 PD-L1⁻/high CD8⁺ patients (11 with 0% PD-L1 expression), 10 patients (7 with 0% PD-L1 expression) had >30% tumor shrinkage, and among 18 PD-L1⁻/low CD8⁺patients (9 with 0% PD-L1 expression), no patients had >30% tumor shrinkage (FIGS. 5A-5D; Table 5). In the combined T1 cohort, ORR among patients with PD-L1⁻ tumors was 29% (95% CI, 8-58); among those with 0% PD-L1 expression, ORR was 40% (95% CI, 12-74; n=10) (Table 6). Among patients in the combined T1 cohort with PD-L1⁺ tumors, ORR was 22% (95% CI, 3-60).

### (c) Pharmacodynamics

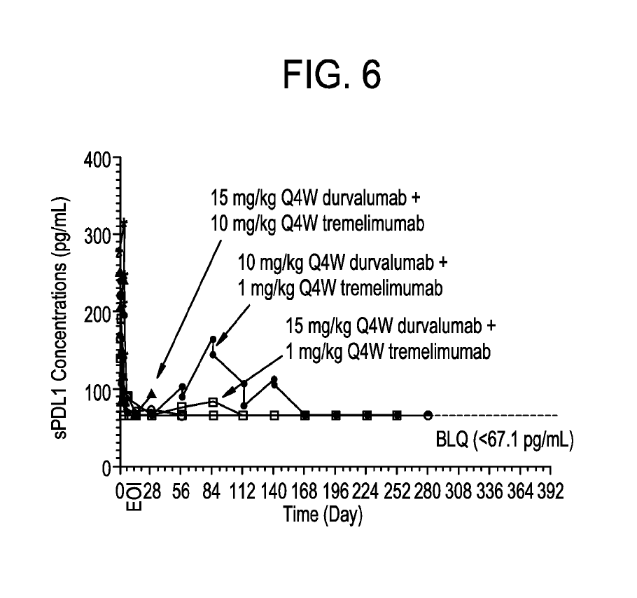

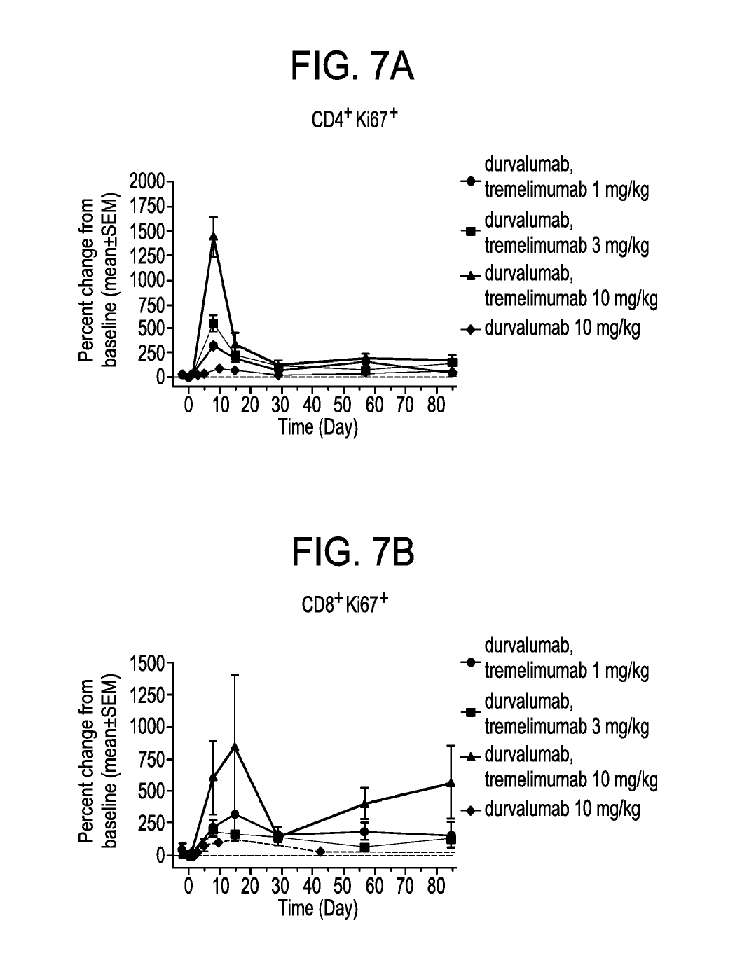

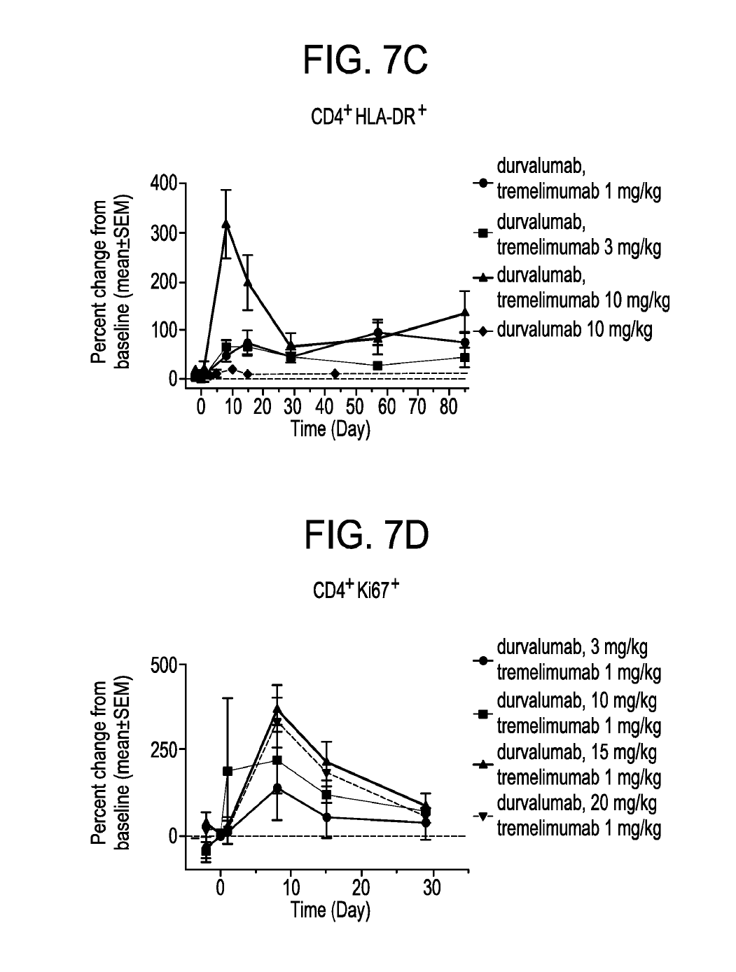

Complete free sPD-L1 suppression was observed in almost all patients across all doses (FIG. 6). A monotonic increase in peak CD4⁺ Ki67⁺ cells was observed with increasing tremelimumab dose (FIGS. 7A-7D). Peak CD8⁺ Ki67⁺ and CD4⁺ HLA-DR⁺ cells were highest with the T10 dose, with T1 and T3 doses eliciting equivalent elevations from baseline. At the lowest tremelimumab dose (1 mg/kg), a trend of durvalumab dose-dependence was observed on mean CD4⁺Ki67⁺ changes from baseline at day 8 and day 15. Combination doses of durvalumab and tremelimumab demonstrated greater peripheral T-cell activation and proliferation than durvalumab monotherapy, even at the lowest tremelimumab dose (1 mg/kg).⁸ Thus, targeting CTLA-4 and PD-L1 was associated with higher biological activity than targeting PD-L1 alone.

### (d) Pharmacokinetics and Immunogenicity

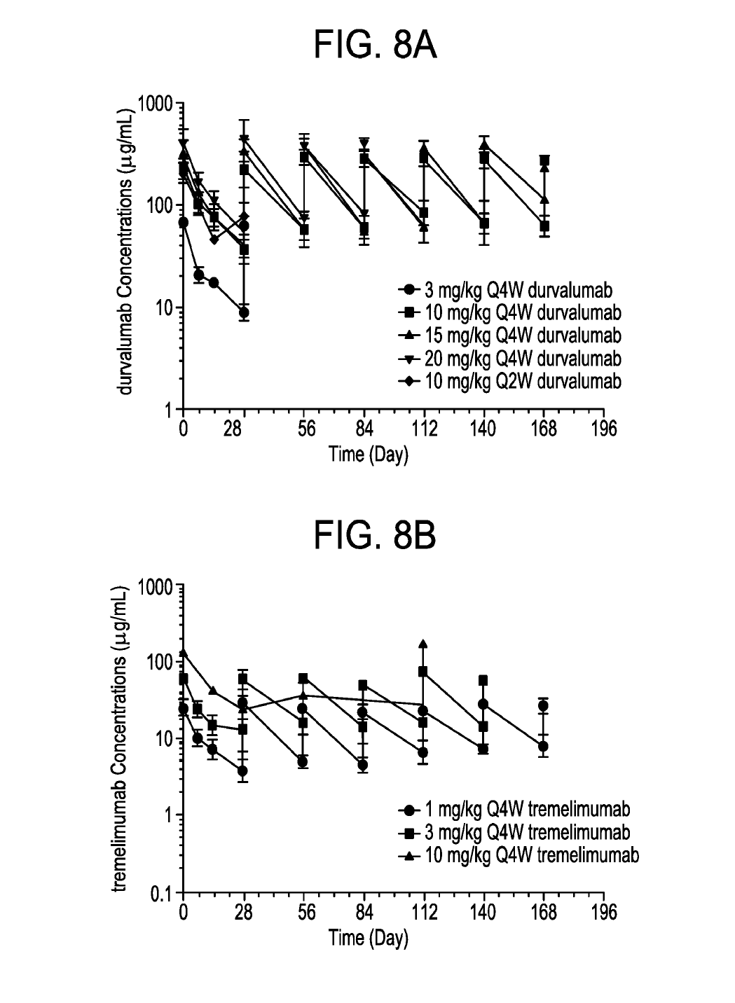

An approximately dose-proportional increase in PK exposure (Cₘₐₓ and AUC_{τ}) of both durvalumab and tremelimumab was observed across all doses (FIGS. 8A and 8B). PK exposures of both durvalumab and tremelimumab in combination following all dosing regimens were in line with monotherapy data^{8.10.17.18} and as predicted by population PK modeling.¹⁵ This indicated no PK interaction between the two drugs. In addition, PK analyses demonstrated that q4w and q2w dosing appeared equivalent.

TABLE 5
Tumor shrinkage by CD8 and PD-L1 status (patients with ≥ 16 weeks follow-up)

| Durvalumab + tremelimumab | | | |
|---|---|---|---|
| All cohorts | | Durvalumab monotherapy (D10 q2w)⁸ | |
| High CD8⁺ | Low CD8⁺ | High CD8⁺ | Low CD8⁺ |

PD-L1⁺ (≥25%)

| | | | | |
|---|---|---|---|---|
| Mean (SEM) | −18.2 (9.0) | 26.0 (26.5) | −21.2 (5.9) | −5.7 (7.1) |
| ≥30% tumor reduction, | 6/15 (40.0) | 0/4 (0) | 18/43 (42) | 8/27 (30) |
| n/N (%) | | | | |
| Objective response, n/N (%)* | 6/14 (43) | 0/5^{†} | 14/45 (31) | 8/33 (24) |

PD-L⁻¹ < 25%

| | | | | |
|---|---|---|---|---|
| Mean (SEM) | −24.8 (7.2) | 9.0 (4.3) | −6.0 (6.7) | 8.2 (5.1) |
| ≥30% tumor reduction, | 10/20 (50) | 0/18 (0) | 6/25 (24) | 3/35 (9) |
| n/N (%) | | | | |
| Objective response, n/N (%)* | 9/20 (45)^{†} | 0/20 (0) | 4/29 (14) | 2/49 (4) |

0%

| | | | | |
|---|---|---|---|---|
| Mean (SEM) | −31.2 (10.5) | 13.8 (4.6) | −15.5 (13.0) | 6.3 (11.7) |
| ≥30% tumor reduction, | 7/11 (64) | 0/9 (0) | 2/6 (33) | 0/8 (0) |
| n/N (%) | | | | |
| Objective response, n/N (%)* | 7/10 (70)^{†} | 0/9 (0) | 1/7 (14) | 0/16 (0) |

*Includes confirmed and unconfirmed responses.
^{†}1 additional patient had stable disease with 27% tumor shrinkage.
SEM, standard error of the mean.

TABLE 6
Response rates by PD-L1 status (confirmed with ≥24 weeks follow-up)

| | Overall population | | | | | | EGFR/ALK wild-type population | | | | | |
|---|---|---|---|---|---|---|---|---|---|---|---|---|
| | | | | Combined T1 cohort | | | | | | Combined T1 cohort | | |
| | | | | D10-20 q4/2w | | | | | | D10-20 q4/2w | | |
| | All cohorts | | | T1* | | | All cohorts | | | T1* | | |
| | n/N (%) | | 95% Cl | n/N (%) | | 95% Cl | n/N (%) | | 95% Cl | n/N (%) | | 95% Cl |
| All patients | 11/63 | (18) | 9-29 | 6/26 | (23) | 9-44 | 11/58 | (19) | 10-31 | 6/25 | (24) | 9-45 |
| PD-L1⁺ (≥25%) | 4/18 | (22) | 6-48 | 2/9 | (22) | 3-60 | 4/15 | (27) | 8-55 | 2/9 | (22) | 3-60 |
| PD-L1⁻ | | | | | | | | | | | | |
| <25% | 6/37 | (16) | 6-32 | 4/14 | (29) | 8-58 | 6/36 | (17) | 6-33 | 4/13 | (31) | 9-61 |
| 0% | 5/24 | (21) | 7-42 | 4/10 | (40) | 12-74 | 5/24 | (21) | 7-42 | 4/10 | (40) | 12-74 |
| All 2L patients | 9/22 | (41) | 21-64 | 5/10 | (50) | 19-81 | 9/22 | (41) | 21-64 | 5/10 | (50) | 19-81 |
| PD-L1⁺ (≥25%) | 3/6 | (50) | 12-88 | 1/3 | (33) | 1-91 | 3/6 | (50) | 12-88 | 1/3 | (33) | 1-91 |
| PD-L1⁻ | | | | | | | | | | | | |
| <25% | 5/12 | (42) | 15-72 | 4/6 | (67) | 22-96 | 5/12 | (42) | 15-72 | 4/6 | (67) | 22-96 |
| 0% | 4/7 | (57) | 18-90 | 4/4 | (100) | 40-100 | 4/7 | (57) | 18-90 | 4/4 | (100) | 40-100 |

2L, second line: 1 prior line of therapy, receiving study treatment in second line
*Excludes D3 q4w T1 cohort (n = 3)

Overall, low levels of ADA were observed following durvalumab (4/60 patients, 6.6%) and tremelimumab (1/53, 1.8%) in combination. In particular, no patient in the D20 q4w/T1 cohort developed ADAs. There was no association between ADAs and tolerability or antitumor activity.

The benefit of single agent PD-1/PD-L1 pathway blockade in a proportion of patients with NSCLC has been clearly demonstrated. However, less than half of NSCLC patients express PD-L1,¹⁶ and the majority of patients (both PD-L1⁺ and PD-L1⁻) do not experience durable benefit from single agent PD-1 pathway blockade, representing an opportunity for combination therapies.

This study is the first to investigate the safety and antitumor activity of durvalumab in combination with the CTLA-4 inhibitor tremelimumab in previously treated patients with locally advanced or metastatic NSCLC.

Employing a unique design to determine an optimal dose, the dose-escalation part of the study demonstrated that combinations of durvalumab with 1 mg/kg tremelimumab had a manageable tolerability profile, and a 1 mg/kg dose of tremelimumab was sufficient to augment the biological and clinical activity of durvalumab. Clinical activity was observed regardless of PD-L1 expression status. The number of CD8⁺ tumor-infiltrating lymphocytes was an important predictor of activity, particularly for the PD-L1 negative population, and could help to identify patients who are more likely to benefit from the combination over monotherapy.

In this study, the MTD was exceeded at D20 q4w/T3. Overall, the combination showed an encouraging tolerability profile at doses of T1 in comparison with higher doses of tremelimumab suggesting a relationship between tremelimumab dose and toxicity. Specifically, doses up to D20 q4w/T1 were well tolerated, with no decrease in antitumor activity compared with the less well tolerated dose of D20 q4w/T3. Rates of treatment-related any Grade and Grade 3/4 AEs were numerically greater with D10 q2w/T1 dosing than with D20 q4w/T1 dosing. The most frequent AEs were consistent with the known toxicity profiles of durvalumab and tremelimumab. The majority of AEs observed were manageable and generally reversible using standard treatment guidelines.

Evidence of antitumor activity was seen with the combination of durvalumab and tremelimumab in patients with advanced NSCLC in the dose-escalation phase of this study, regardless of PD-L1 status. In comparison, ORR in patients with PD-L1⁻ tumors receiving 10 mg/kg q2w durvalumab monotherapy was 5%.⁸ Activity was notable among patients with PD-L1⁻ tumors, particularly those patients who fell well below the cutoff of 25%, including those with 0% PD-L1 expression. Specifically, in the combined T1 cohort, ORR in patients with PD-L1⁻ tumors was 29% (95% CI, 8-58); neither ORR nor DCR was greater in the combined T3 cohort or the T10 cohort. Without being bound to theory, this indicates that PD-L1 status may not predict response to the durvalumab and tremelimumab combination to the same extent as has been seen with durvalumab monotherapy.

The presence of high numbers of CD8⁻ tumor-infiltrating lymphocytes in these patients indicates that CD8 status may be an important predictor of activity and could better identify patients who are more likely to benefit from the combination over monotherapy. This observation also indicates that additional factors beyond PD-L1 are involved in suppressing an active immune response. Without being bound to theory, CTLA-4 activity may prevail in such patients and tremelimumab removes a suppressive effect to drive an antitumor response. The antitumor activity of the combination appears to be higher than that of monotherapy with either agent,^{8.17} most likely because they influence distinct targets involved in immunosuppression, acting on different aspects of the antitumor immune response. Previous studies in NSCLC and other tumor types have also indicated that combined blockade of PD-1 and CTLA-4 is associated with higher clinical activity than monotherapy.^{3,19-22}

The results of this study show that toxicity, but not antitumor activity, tended to increase with increasing doses of tremelimumab. As there were no pharmacological limitations evident with the q4w schedule, and given the equivalent PK profiles seen with D20 q4w and D10 q2w, q4w was selected over the q2w schedule for patient convenience. The D20 q4w/T1 regimen has therefore been selected for assessment in Phase 3 studies. This dose maximizes free sPD-L1 inhibition, has a manageable safety profile, and incorporates a biologically active dose of tremelimumab that is associated with antitumor activity, including in patients with PD-L1⁻ tumors. Doses above T1 did not result in greater antitumor activity but were generally associated with higher AE rates.

The clinical activity in patients with PD-L1⁻ tumors, including those with no tumor cell membrane PD-L1 staining, is a particularly important advance, as these patients are less responsive to single agents blocking the PD-1 checkpoint pathway. On the basis of these investigations, the dose of combination treatment with durvalumab and tremelimumab was selected for Phase 3 studies

In conclusion, the tolerability profile and antitumor activity of the combination observed both in PD-L1+ and PD-L1⁻ patients in the dose-escalation phase of this study shows that 1 mg/kg tremelimumab is sufficient to augment the biological and antitumor activity of durvalumab.

Those skilled in the art will recognize, or be able to ascertain using no more than routine experimentation, many equivalents to the specific aspects of the disclosure described herein. Such equivalents are intended to be encompassed by the following claims.

Although the foregoing invention has been described in some detail by way of illustration and example for purposes of clarity of understanding, it will be obvious that certain changes and modifications can be practiced within the scope of the appended claims.

Various publications are cited herein, the disclosures of which are incorporated by reference in their entireties.

The following references are cited herein:

### References

1. Gajewski T F, Schreiber H, Fu Y X. Innate and adaptive immune cells in the tumor microenvironment. Nat Immunol 2013; 14: 1014-22.
2. Kvistborg P, Philips D, Kelderman S, et al. Anti-CTLA-4 therapy broadens the melanoma-reactive CD8+ T cell response. Sci Transl Med 2014; 6:254ra128.
3. Larkin J, Chiarion-Sileni V, Gonzalez R, et al. Combined nivolumab and ipilimumab or monotherapy in untreated melanoma. N Engl J Med 2015; 373: 23-34.
4. Antonia S J, Gettinger S N, Chow L Q M, et al. Nivolumab (anti-PD-1; BMS-936558, ONO-4538) and ipilimumab in first-line NSCLC: Interim phase I results. J Clin Oncol 2014; 32: Supp1:8023. abstract.
5. MedImmune/AstraZeneca, Data on file. 2015.
6. Matsumoto K, Fukuyama S, Eguchi-Tsuda M, et al. B7-DC induced by IL-13 works as a feedback regulator in the effector phase of allergic asthma. Biochem Biophys Res Commun 2008; 365: 170-5.
7. Matsumoto K, Inoue H, Nakano T, et al. B7-DC regulates asthmatic response by an IFN-gamma-dependent mechanism. J Immunol 2004; 172: 2530-41.
8. Rizvi N, Brahmer J, Ou S-HI. Safety and clinical activity of MEDI4736, an anti-programmed cell death-ligand 1 (PD-L1) antibody, in patients with non-small cell lung cancer (NSCLC). J Clin Oncol 2015; 33: Supp1:8032. abstract.
9. Ribas A, Camacho L H, Lopez-Berestein G, et al. Antitumor activity in melanoma and anti-self responses in a phase I trial with the anti-cytotoxic T lymphocyte-associated antigen 4 monoclonal antibody CP-675,206. J Clin Oncol 2005; 23: 8968-77.
10. Tarhini A A. Tremelimumab: a review of development to date in solid tumors. Immunotherapy 2013; 5: 215-29.
11. Stewart R, Mullins S, Watkins A, et al. Preclinical modeling of immune checkpoint blockade (P2012). J Immunol 2013; 190: Supp1:214.7.
12. Huang X, Biswas S, Oki Y, Issa J P, Berry D A. A parallel phase I/II clinical trial design for combination therapies. Biometrics 2007; 63: 429-36.
13. Eisenhauer E A, Therasse P, Bogaerts J, et al. New response evaluation criteria in solid tumours: revised RECIST guideline (version 1.1). Eur J Cancer 2009; 45: 228-47.
14. Rebelatto M, Mistry A, Sabalos C, et al. Development of a PD-L1 companion diagnostic assay for treatment with MEDI4736 in NSCLC and SCCHN patients. J Clin Oncol 2015; 33:Suppl: 8033. abstract.
15. Song X, Pak M, Chavez C, et al. Population pharmacokinetics of MEDI4736, a fully human anti-programmed death ligand 1 (PD-L1) monoclonal antibody, in patients with advanced solid tumors. European Cancer Congress 2015 Sep 25-29; Vienna, Austria: ECC; 2015. Abstract 203.
16. Pan Z K, Ye F, Wu X, An H X, Wu J X. Clinicopathological and prognostic significance of programmed cell death ligand1 (PD-L1) expression in patients with non-small cell lung cancer: a meta-analysis. J Thorac Dis 2015; 7: 462-70.
17. Zatloukal P, Heo DS, Park K, et al. Randomized phase II clinical trial comparing tremelimumab (CP-675,206) with best supportive care (BSC) following first-line platinum-based therapy in patients (pts) with advanced non-small cell lung cancer (NSCLC). J Clin Oncol 2009; 27:Suppl: 8071. abstract.
18. Lutzky J, Antonia S J, Blake-Haskins A, et al. A phase 1 study of MEDI4736, an anti-PD-L1 antibody, in patients with advanced solid tumors. J Clin Oncol 2014; 32:Suppl: 3001. abstract.
19. Antonia S J, Gettinger S N, Chow L, et al. Nivolumab (anti-PD-1; BMS-936558, ONO-4538) and ipilimumab in first-line NSCLC: Interim phase I results. J Clin Oncol 2014;32: Suppl: 8023. abstract.
20. Antonia S J, Larkin J, Ascierto P A. Immuno-oncology combinations: a review of clinical experience and future prospects. Clin Cancer Res 2014; 20: 6258-68.
21. Swanson M S, Sinha U K. Rationale for combined blockade of PD-1 and CTLA-4 in advanced head and neck squamous cell cancer-review of current data. Oral Oncol 2015; 51: 12-5.
22. Wolchok J D, Kluger H, Callahan M K, et al. Nivolumab plus ipilimumab in advanced melanoma. N Engl J Med 2013; 369: 122-33.

What is claimed:

1. A method of treatment comprising administering durvalumab, or an antigen-binding fragment thereof, and tremelimumab, or an antigen-binding fragment thereof, to a patient identified as having PD-L1- non-small cell lung cancer (NSCLC) comprising a high level of CD8+tumor-infiltrating lymphocytes.

2. The method of claim 1, wherein the level of CD8+ tumor-infiltrating lymphocytes is greater than about 300-350 cells/mm².

3. The method of claim 2, wherein the level of CD8+ tumor-infiltrating lymphocytes is greater than about 300-325 cells/mm².

4. The method of claim 3, wherein the level of CD8+ tumor-infiltrating lymphocytes is greater than about 317 cells/mm².

5. The method of claim 1, wherein the level of CD830 tumor-infiltrating lymphocytes is measured prior to treatment.

6. The method of claim 1, wherein the level of CD8+ tumor-infiltrating lymphocytes is measured in a tumor biopsy.

7. The method of claim 1, wherein the administration results in a decrease in tumor size as compared to the administration of durvalumab, or an antigen-binding fragment thereof, alone.

8. The method of claim 5, wherein the administration reduces tumor size by at least about 30% or more relative to baseline.

9. The method of claim 5, wherein the administration results in an increase in objective response rate as compared to the administration of durvalumab, or an antigen-binding fragment thereof, alone.

10. The method of claim 1, wherein the durvalumab, or antigen-binding fragment thereof, is administered at 3, 10, 15, or 20 mg/kg.

11. The method of claim 1, wherein the tremelimumab, or antigen-binding fragment thereof, is administered at 1, 3, or 10 mg/kg.

12. The method of claim 1, wherein the durvalumab, or antigen-binding fragment thereof, is administered at 20 mg/kg and the tremelimumab, or antigen-binding fragment thereof, is administered at 1 mg/kg.

13. The method of claim 1, wherein the durvalumab, or antigen-binding fragment thereof, is administered at 20 mg/kg every 4 weeks and the tremelimumab, or antigen-binding-fragment thereof, is administered at 1 mg/kg.

14. The method of claim 1, wherein the durvalumab, or antigen-binding fragment thereof, is administered every 2 weeks.

15. The method of claim 1, wherein the durvalumab, or antigen-binding fragment thereof is administered every 4 weeks.

16. The method of claim 1, wherein the durvalumab, or antigen-binding fragment thereof, and tremelimumab, or antigen-binding fragment thereof, are administered concurrently.

17. The method of claim 1, wherein the durvalumab, or antigen-binding fragment thereof, is administered by intravenous injection.

18. The method of claim 1, wherein the tremelimumab, or antigen-binding fragment thereof, is administered by intravenous injection.
